(19) United States
(12) Patent Application Publication
(10) Pub. No.: US 2010/0137561 A1
(43) Pub. Date: Jun. 3, 2010

(54) PROCESS FOR PREPARING THERAPEUTIC PEPTIDE

(76) Inventor: Lin Chen, Superior, CO (US)

Correspondence Address: Grant D Green;Patent Law Department, M/S A2-250, Roche Palo Alto LLC, 3431 Hillview Avenue, Palo Alto, CA, 94304, US

(21) Appl. No.: 12/630,002

(22) Filed: Dec. 3, 2009

Related U.S. Application Data
(60) Provisional application No. 61/200,848, filed on Dec. 3, 2008.

(51) Int. Cl. C07K 14/435; C07K 7/08; C07K 7/06; C07K 5/103

(52) U.S. Cl. 530/324; 530/326; 530/327; 530/330

(57) ABSTRACT

This application discloses processes for synthesizing human relaxin Chain B for treatment of diseases mediated by relaxin. This application in particular discloses processes of synthesizing the Chain B of human relaxin using a solid and solution phase (“hybrid”) approach. Generally, the approach includes synthesizing three different peptide intermediate fragments using solid phase chemistry. Solution phase chemistry is then used to couple the fragments.

### CROSS-REFERENCE TO RELATED APPLICATIONS

This application is entitled to the benefit of U.S. provisional patent application Ser. No. 61/200,848 filed on Dec. 3, 2008, the disclosure of which is incorporated herein by reference.

### FIELD OF THE INVENTION

This invention is in the field of peptide synthesis related to treatment of diseases mediated by human relaxin, such as vasoconstriction, and in particular to the process of synthesizing the Chain B of relaxin and use thereof in the treatment of diseases mediated by relaxin.

### BACKGROUND OF THE INVENTION

Relaxin (RLX) is a low molecular weight protein of approximately 6,000 Da belonging to the insulin-growth factor family that circulates during the luteal phase of the menstrual cycle and throughout gestation in women. It is also produced by the prostate in men. RLX is also a pregnancy hormone in rats. In both species, circulating levels derive from the corpus luteum. Relaxin consists of two peptide chains, referred to as A and B, joined by disulfide bonds with an intra-chain disulfide loop in the A-chain in a manner analogous to that of insulin. Relaxin is synthesized in the corpora lutea of ovaries during pregnancy, and is released into the blood stream prior to parturition. The availability of ovarian tissue has enabled the isolation and amino acid sequence determination of relaxin from the pig (James et al (1977), Nature, 267, 554-546), the rat (John et al (1981) Endocrinology, 108, 726-729), and the shark (Schwabe et al (1982) Ann. N.Y. Acad. Sci., 380, 6-12).

Three separate human relaxin genes have been identified and designated as H1 (Hudson et al (1983) Nature, 301, 628-631), H2 (Hudson et al (1984) Embo. J, 3, 2333-2339), and H3 (R. A. Bathgate et al. J. Biol. Chem. 277 (2002), pp. 1148-1157). The peptide encoded by the H2 gene is referred to as “relaxin” as it is the major stored and circulating form in the human (Winslow et al (1992) Endocrinology, 130, 2660-2668).

Evidence has accumulated to suggest that relaxin is more than a hormone of pregnancy and acts on cells and tissues other than those of the female reproductive system (E. D. Lekgabe et al., Endocrinology 147 (2006), pp. 5575-5583; I. Mookerjee et al., Endocrinology 147 (2006), pp. 754-761). Relaxin causes a widening of blood vessels (vasodilatation) in the kidney, mesocaecum, lung and peripheral vasculature, which leads to increased blood flow or perfusion rates in these tissues and stimulates an increase in heart rate and coronary blood flow, and increases both glomerular filtration rate and renal plasma flow (T. D. Hewitson et al., Endocrinology 148 (2007), pp. 660-669; Bani et al (1997) Gen. Pharmacol. 28, 13-22). The brain is another target tissue for relaxin where the peptide has been shown to bind to receptors (Osheroff et al (1991) Proc. NaI. Acad. Sci. U.S.A. 88, 6413-6417; Tan et al (1999) Br. J. Pharmacol 127, 91-98) in the circumventricular organs to affect blood pressure and drinking (Parry et al (1990) J Neurodendocrinol 2, 53-58; Summerlee et al (1998) Endocrinology 139, 2322-2328; Sinnahay et al (1999) Endocrinology 140, 5082-5086).

Important clinical uses arise for relaxin in various diseases responding to vasodilation, such as coronary artery disease, peripheral vascular disease, kidney disease associated with arteriosclerosis or other narrowing of kidney capillaries, or other capillaries narrowing in the body, such as in the eyes or in the peripheral digits, the mesocaecum, lung and peripheral vasculature (C. S. Samuel et al., Pharmacol. Ther. 112 (2006), pp. 529-552; S, Nistri et al., Cardiovasc. Hematol. Agents Med. Chem. 5 (2007), pp. 101-108; D. Bani and M. Bigazzi, Curr. Med. Chem.-IEMA 5 (2005), pp. 403-410; T. Dschietzig et al., Pharmacol. Ther. 112 (2006), pp. 38-56).

In view of the ongoing problems associated with hypertensive vascular disease, it is clear that there is a need in the art for additional means of treating hypertensive vascular disease. The present invention addresses this need and provides related advantages as well.

### SUMMARY OF THE INVENTION

This application provides processes for synthesizing human relaxin Chain B for treatment of diseases mediated by relaxin. This application in particular discloses processes of synthesizing the Chain B of human relaxin using a solid and solution phase (“hybrid”) approach. Generally, the approach includes synthesizing three different peptide intermediate fragments using solid phase chemistry. Solution phase chemistry is then used to couple the fragments.

In one aspect, the application provides a process for preparing a relaxin Chain B peptide comprising the step of:

a) introducing a peptide fragment including the amino acid sequence of

(SEQ ID NO. 3)

Z-Met-Ser-Thr-Trp-OH

wherein:

### Z is H—; and

one or more residues of said sequence optionally include side chain protection.

The application also provides the above process, further comprising the steps of:

b) coupling the peptide fragment of step a) in solution to H-Ser-OtBu in order to provide a peptide fragment including the amino acid sequence of

(SEQ ID NO. 4)

Z-Met-Ser-Thr-Trp-Ser-OtBu

wherein:
Z is N-terminal protecting group Fmoc-; and
one or more residues of said sequence optionally include side chain protection; and
c) removing the N-terminal protecting group to afford a peptide fragment including the amino acid sequence of

(SEQ ID NO. 4)

Z-Met-Ser-Thr-Trp-Ser-OtBu

wherein:

### Z is H; and

one or more residues of said sequence optionally include side chain protection.

The application also provides the above process, further comprising the steps of:

d) introducing a peptide fragment including the amino acid sequence of

(SEQ ID NO. 2)

Z-Arg-Glu-Leu-Val-Arg-Ala-Gln-Ile-Ala-Ile-Cys-Gly-
OH

wherein:
Z is N-terminal protecting group Fmoc-; and
one or more residues of said sequence optionally include side chain protection;
e) coupling the peptide fragment of step d) in solution to the peptide fragment of step c) in the presence of an alkali metal halide in order to provide a peptide fragment including the amino acid sequence of

(SEQ ID NO. 5)

Z-Arg-Glu-Leu-Val-Arg-Ala-Gln-Ile-Ala-Ile-Cys-Gly-
Met-Ser-Thr-Trp-Ser-OtBu

wherein:
Z is N-terminal protecting group Fmoc-; and
one or more residues of said sequence optionally include side chain protection; and
f) removing the N-terminal protecting group to afford a peptide fragment including the amino acid sequence of

(SEQ ID NO. 5)

Z-Arg-Glu-Leu-Val-Arg-Ala-Gln-Ile-Ala-Ile-Cys-Gly-
Met-Ser-Thr-Trp-Ser-OtBu

wherein:

### Z is H; and

one or more residues of said sequence optionally include side chain protection.

The application also provides the above process, further comprising the steps of:

g) introducing a peptide fragment including the amino acid sequence of

(SEQ ID NO. 1)

Z-Asp-Ser-Trp-Met-Glu-Glu-Val-Ile-Lys-Leu-Cys-Gly-
OH

wherein:
Z is N-terminal protecting group Boc-; and
one or more residues of said sequence optionally include side chain protection; and
h) coupling the peptide fragment of step f) in solution to the peptide fragment of step g) in the presence of an alkali metal halide in order to provide a peptide including the amino acid sequence of

(SEQ ID NO. 6)

Z-Asp-Ser-Trp-Met-Glu-Glu-Val-Ile-Lys-Leu-Cys-Gly-
Arg-Glu-Leu-Val-Arg-Ala-Gln-Ile-Ala-Ile-Cys-Gly-
Met-Ser-Thr-Trp-Ser-OtBu

wherein:
Z is N-terminal protecting group Boc-; and
one or more residues of said sequence optionally include side chain protection.

The application also provides the above process, further comprising the step of:

i) contacting the peptide resulting from step h) with acid in order to remove the N-terminal protecting group and deprotect the amino acid side chains to afford the deprotected relaxin Chain B amino acid sequence of

(SEQ ID NO. 7)

Z-Asp-Ser-Trp-Met-Glu-Glu-Val-Ile-Lys-Leu-Cys-Gly-
Arg-Glu-Leu-Val-Arg-Ala-Gln-Ile-Ala-Ile-Cys-Gly-
Met-Ser-Thr-Trp-Ser-OH

wherein:

### Z is H.

In one aspect, the application provides a process for preparing a relaxin Chain B peptide comprising the step of:

a) introducing a peptide fragment including the amino acid sequence of

(SEQ ID NO. 3)

Z-Met-Ser(OtBu)-Thr(OtBu)-Trp-OH

wherein:
Z is N-terminal protecting group Fmoc-.

The application also provides the above process, further comprising the step of:

b) coupling the peptide fragment of step a) in solution to H-Ser(tBu)-OtBu in order to provide a peptide fragment including the amino acid sequence of

(SEQ ID NO. 4)

Z-Met-Ser(OtBu)-Thr(OtBu)-Trp-Ser(OtBu)-OtBu

wherein:
Z is N-terminal protecting group Fmoc-.

The application also provides the above process, further comprising the steps of:

c) removing the N-terminal protecting group to afford a peptide fragment including the amino acid sequence of

(SEQ ID NO. 4)

Z-Met-Ser(OtBu)-Thr(OtBu)-Trp-Ser(OtBu)-OtBu

wherein:

### Z is H;

d) introducing a peptide fragment including the amino acid sequence of

(SEQ ID NO. 2)

Z-Arg(Pbf)-Glu(OtBu)-Leu-Val-Arg(Pbf)-Ala-Gln
(Trt)-Ile-Ala-Ile-Cys(Trt)-Gly-OH

wherein:
Z is N-terminal protecting group Fmoc-; and
e) coupling the peptide fragment of step c) in solution to the fragment of step d) in the presence of an alkali metal halide in order to provide a peptide fragment including the amino acid sequence of

(SEQ ID NO. 5)

Z-Arg(Pbf)-Glu(OtBu)-Leu-Val-Arg(Pbf)-Ala-Gln
(Trt)-Ile-Ala-Ile-Cys(Trt)-Gly-Met-Ser(OtBu)-Thr
(OtBu)-Trp-Ser(OtBu)-OtBu

wherein:
Z is N-terminal protecting group Fmoc-; and
f) removing the N-terminal protecting group to afford a peptide fragment including the amino acid sequence of

(SEQ ID NO. 5)

Z-Arg(Pbf)-Glu(OtBu)-Leu-Val-Arg(Pbf)-Ala-Gln
(Trt)-Ile-Ala-Ile-Cys(Trt)-Gly-Met-Ser(OtBu)-Thr
(OtBu)-Trp-Ser(OtBu)-OtBu

wherein:

### Z is H.

The application also provides the above process, further comprising the steps of:

g) introducing a peptide fragment including the amino acid sequence of

(SEQ. ID NO. 1)

Z-Asp(OtBu)-Ser(tBu)-Trp(Boc)-Met-Glu(OtBu)-Glu
(OtBu)-Val-Ile-Lys(Boc)-Leu-Cys(Trt)-Gly-OH

wherein:
Z is N-terminal protecting group Boc-;
h) coupling the peptide fragment of step f) in solution to the peptide fragment of step g) in the presence of an alkali metal halide in order to provide a peptide including the amino acid sequence of

(SEQ ID NO. 6)

Z-Asp(OtBu)-Ser(tBu)-Trp(Boc)-Met-Glu(OtBu)-Glu
(OtBu)-Val-Ile-Lys(Boc)-Leu-Cys(Trt)-Gly-Arg(Pbf)-
Glu(OtBu)-Leu-Val-Arg(Pbf)-Ala-Gln(Trt)-Ile-Ala-
Ile-Cys(Trt)-Gly-Met-Ser(OtBu)-Thr(OtBu)-Trp-Ser
(OtBu)-OtBu

wherein:
Z is N-terminal protecting group Boc-;
i) contacting the peptide resulting from step h) with acid in order to remove the N-terminal protecting group and deprotect the amino acid side chains to afford the deprotected relaxin Chain B amino acid sequence of

(SEQ ID NO. 7)

Z-Asp-Ser-Trp-Met-Glu-Glu-Val-Ile-Lys-Leu-Cys-Gly-
Arg-Glu-Leu-Val-Arg-Ala-Gln-Ile-Ala-Ile-Cys-Gly-
Met-Ser-Thr-Trp-Ser-OH

wherein:

### Z is H.

In one aspect, the application provides a process for preparing a relaxin Chain B peptide comprising the step of:

a) introducing a peptide fragment including the amino acid sequence of

(SEQ ID NO. 2)

Fmoc-Arg(Pbf)-Glu(OtBu)-Leu-Val-Arg(Pbf)-Ala-Gln
(Trt)-Ile-Ala-Ile-Cys(Trt)-Gly-OH

wherein:
Z is N-terminal protecting group Fmoc-;
b) introducing a peptide fragment including the amino acid sequence of

(SEQ ID NO. 4)

H-Met-Ser(OtBu)-Thr(OtBu)-Trp-Ser(OtBu)-OtBu

wherein:

### Z is H;

c) coupling the peptide fragment of step a) in solution to the fragment of step b) in the presence of an alkali metal halide in order to provide a peptide fragment including the amino acid sequence of

(SEQ ID NO. 5)

Z-Arg(Pbf)-Glu(OtBu)-Leu-Val-Arg(Pbf)-Ala-Gln
(Trt)-Ile-Ala-Ile-Cys(Trt)-Gly-Met-Ser(OtBu)-Thr
(OtBu)-Trn-Ser(OtBu)-OtBu

wherein:
Z is N-terminal protecting group Fmoc-;
d) removing the N-terminal protecting group to afford a peptide fragment including the amino acid sequence of

(SEQ ID NO. 5)

Z-Arg(Pbf)-Glu(OtBu)-Leu-Val-Arg(Pbf)-Ala-Gln
(Trt)-Ile-Ala-Ile-Cys(Trt)-Gly-Met-Ser(OtBu)-Thr
(OtBu)-Trp-Ser(OtBu)-OtBu

wherein:

### Z is H.

In one aspect, the application provides a process for preparing a relaxin Chain B peptide comprising the step of:

a) introducing a peptide fragment including the amino acid sequence of

(SEQ ID NO. 5)

Z-Arg(Pbf)-Glu(OtBu)-Leu-Val-Arg(Pbf)-Ala-Gln
(Trt)-Ile-Ala-Ile-Cys(Trt)-Gly-Met-Ser(OtBu)-Thr
(OtBu)-Trp-Ser(OtBu)-OtBu

wherein:

### Z is H;

b) introducing a peptide fragment including the amino acid sequence of

(SEQ. ID NO. 1)

Z-Asp(OtBu)-Ser(tBu)-Trp(Boc)-Met-Glu(OtBu)-Glu
(OtBu)-Val-Ile-Lys(Boc)-Leu-Cys(Trt)-Gly-OH

wherein:
Z is N-terminal protecting group Boc-;
c) coupling the peptide fragment of step a) in solution to the peptide fragment of step b) in the presence of an alkali metal halide in order to provide a peptide including the amino acid sequence of

(SEQ ID NO. 6)

Z-Asp(OtBu)-Ser(tBu)-Trp(Boc)-Met-Glu(OtBu)-Glu
(OtBu)-Val-Ile-Lys(Boc)-Leu-Cys(Trt)-Gly-Arg(Pbf)-
Glu(OtBu)-Leu-Val-Arg(Pbf)-Ala-Gln(Trt)-Ile-Ala-
Ile-Cys(Trt)-Gly-Met-Ser(OtBu)-Thr(OtBu)-Trp-Ser
(OtBu)-OtBu

wherein:
Z is N-terminal protecting group Boc.

In one aspect, the application provides a peptide of the amino acid sequence

(SEQ. ID NO. 1)

Z-Asp(OtBu)-Ser(tBu)-Trp(Boc)-Met-Glu(OtBu)-Glu
(OtBu)-Val-Ile-Lys(Boc)-Leu-Cys(Trt)-Gly-OH

wherein:
Z is H or N-terminal protecting group Boc.

In one aspect, the application provides a peptide of the amino acid sequence

(SEQ. ID NO.2)

Z-Arg(Pbf)-Glu(OtBu)-Leu-Val-Arg(Pbf)-Ala-Gln
(Trt)-Ile-Ala-Ile-Cys(Trt)-Gly-OH

wherein:
Z is H or N-terminal protecting group Fmoc.

In one aspect, the application provides a peptide of the amino acid sequence

(SEQ. ID NO. 3)

Z-Met-Ser(OtBu)-Thr(OtBu)-Trp-OH

wherein:
Z is H or N-terminal protecting group Fmoc.

In one aspect, the application provides a peptide prepared by the process of claim 4 comprising the amino acid sequence

(SEQ ID NO. 4)

Z-Met-Ser(OtBu)-Thr(OtBu)-Trp-Ser(OtBu)-OtBu

wherein:
Z is H or N-terminal protecting group Fmoc.

In one aspect, the application provides a peptide of the amino acid sequence

(SEQ ID NO. 5)

Z-Arg(Pbf)-Glu(OtBu)-Leu-Val-Arg(Pbf)-Ala-Gln
(Trt)-Ile-Ala-Ile-Cys(Trt)-Gly-Met-Ser(OtBu)-Thr
(OtBu)-Trp-Ser(OtBu)-OtBu

wherein:
Z is H or N-terminal protecting group Fmoc.

In one aspect, the application provides a peptide of the amino acid sequence

(SEQ ID NO. 6)

Z-Asp(OtBu)-Ser(tBu)-Trp(Boc)-Met-Glu(OtBu)-Glu
(OtBu)-Val-Ile-Lys(Boc)-Leu-Cys(Trt)-Gly-Arg(Pbf)-
Glu(OtBu)-Leu-Val-Arg(Pbf)-Ala-Gln(Trt)-Ile-Ala-
Ile-Cys(Trt)-Gly-Met-Ser(OtBu)-Thr(OtBu)-Trp-Ser
(OtBu)-OtBu

wherein:
Z is H or N-terminal protecting group Boc or Fmoc.

### DETAILED DESCRIPTION

The embodiments of the present invention described herein are not intended to be exhaustive or to limit the invention to the precise forms disclosed in the following detailed description. Rather, the embodiments are chosen and described so that others skilled in the art can appreciate and understand the principles and practices of the present invention.

The present invention related to a novel process to synthesize the relaxin Chain B peptide. The relaxin Chain B peptide has the following formula

(SEQ. ID NO. 7):
H-Asp-Ser-Trp-Met-Glu-Glu-Val-Ile-Lys-Leu-Cys-Gly-
Arg-Glu-Leu-Val-Arg-Ala-Gln-Ile-Ala-Ile-Cys-Gly-
Met-Ser-Thr-Trp-Ser-OH

### DEFINITIONS

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

As used in this specification, whether in a transitional phrase or in the body of the claim, the terms “comprise(s)” and “comprising” are to be interpreted as having an open-ended meaning That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which R appears twice and is defined as “independently carbon or nitrogen”, both R's can be carbon, both R's can be nitrogen, or one R can be carbon and the other nitrogen.

When any variable occurs more than one time in any moiety or formula depicting and describing compounds employed or claimed in the present invention, its definition on each occurrence is independent of its definition at every other occurrence. Also, combinations of substituents and/or variables are permissible only if such

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen or a substituent.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, the term “including the amino acid sequence” preferably means “having the amino acid sequence”.

As used herein, the term “alkali metal halide” means a salt comprising an alkali metal ion such as Li⁺ or Cs⁺ and a halide ion such as F⁻, Cl⁻, Br⁻, or I⁻. In a preferred embodiment, the alkali metal halide is LiBr.

Generally, the amino acids from which peptides are derived can be naturally occurring amino acid residues, non-natural amino acid residues, or combinations thereof. The twenty common naturally-occurring amino acid residues are as follows: A (Ala, alanine), R (Arg, arginine); N (Asn, asparagine); D (Asp, aspartic acid); C (Cys, cysteine) Q (Gln, glutamine), E (Glu, glutamic acid); G (Gly, glycine); H (His, histidine); I (Ile, isoleucine); L (Leu, leucine); K (Lys, lysine); M (Met, methionine); F (Phe, phenylalanine); P (Pro, proline); S (Ser, serine); T (Thr, threonine); W (Trp, tryptophan); Y (Tyr, tyrosine); and V (Val, valine).

The nature and use of protecting groups is well known in the art. Generally, a suitable protecting group is any sort of group that can help prevent the atom to which it is attached, typically oxygen or nitrogen, from participating in undesired reactions during processing and synthesis. Protecting groups include side chain protecting groups and amino- or N-terminal protecting groups. Protecting groups can also prevent reaction or bonding of carboxylic acids, thiols, and the like.

A side chain protecting group refers to a chemical moiety coupled to the side chain (R group in the general amino acid formula H2N—C(R)(H)—COOH) of an amino acid that helps prevent a portion of the side chain from reacting with chemicals used in steps of peptide synthesis, processing, and the like. The choice of a side chain protecting group can depend upon various factors, for example, the type of synthesis performed, processing to which the peptide will be subjected, and the desired intermediate product or final product. The side chain protecting group also depends upon the nature of the amino acid itself. Generally, a side chain protecting group is chosen that is not removed during deprotection of the alpha-amino groups during synthesis. Therefore, the alpha-amino protecting group and the side chain protecting group are typically not the same.

In some cases, and depending upon the type of reagents used in solid phase synthesis and other peptide processing, an amino acid may not require the presence of a side chain protecting group. Such amino acids typically do not include a reactive oxygen or nitrogen in the side chain.

Examples of side chain protecting groups include acetyl (Ac), benzoyl (Bz), tert butyl, triphenylmethyl (trityl), tetrahydropyranyl, benzyl ether (Bzl), 2,6-dichlorobenzyl (DCB), t-butoxycarbonyl (BOC), 2,2,4,6,7-pentamethyldihydrobenzofuran-5-sulfonamide (Pbf), nitro, p-toluenesulfonyl (Tos), adamantyloxycarbonyl, xanthyl (Xan), benzyl, methyl, ethyl, and t-butyl ester, benzyloxycarbonyl (Z), 2-chlorobenzyloxycarbonyl (2-Cl-Z), t-amyloxycarbonyl (Aoc), and aromatic or aliphatic urethan-type protecting groups, photolabile groups such as nitro veratryl oxycarbonyl (NVOC), and fluoride labile groups such as trimethylsilylethyl oxycarbonyl (TEOC).

For example, side chains of the amino acid residues of peptide fragments can be protected with standard protecting groups such as OtButyl (OtBu), t-butyl (t-Bu), trityl (trt), and t-butyloxycarbonyl (Boc). In the present invention, preferred side chain protecting groups include the OtBu group for Asp, Glu, and Thr, the tBu group and the OtBu group for Ser, the Trt group for Cys and Gln, the Pbf group for Arg, and the Boc group for Trp and Lys.

An amino terminal protecting group includes a chemical moiety coupled to the alpha amino group of an amino acid. Typically, the amino-terminal protecting group is removed in a deprotection reaction prior to the addition of the next amino acid to be added to the growing peptide chain, but can be maintained when the peptide is cleaved from the support. The choice of an amino terminal protecting group can depend upon various factors, for example, the type of synthesis performed and the desired intermediate product or final product obtained.

Examples of amino terminal protecting groups include: (1) acyl-type protecting groups, such as formyl, acryloyl (Acr), benzoyl (Bz) and acetyl (Ac); (2) aromatic urethan-type protecting groups, such as benzyloxycarbonyl (Z) and substituted Z, such as p-chlorobenzyloxycarbonyl, p-nitrobenzyloxycarbonyl, p-bromobenzyloxycarbonyl, p-methoxybenzyloxycarbonyl; (3) aliphatic urethan protecting groups, such as t-butyloxycarbonyl (BOC), diisopropylmethoxycarbonyl, isopropyloxycarbonyl, ethoxycarbonyl, allyloxycarbonyl; (4) cycloalkyl urethan-type protecting groups, such as 9-fluorenylmethyloxycarbonyl (Fmoc), cyclopentyloxycarbonyl, adamantyloxycarbonyl, and cyclohexyloxycarbonyl; and (5) thiourethan-type protecting groups, such as phenylthiocarbonyl. Preferred protecting groups include 9-fluorenylmethyloxycarbonyl (Fmoc), 2-(4-biphenylyl)-propyl(2)oxycarbonyl (Bpoc), 2-phenylpropyl(2)-oxycarbonyl (Poc), and t-butyloxycarbonyl (Boc).

Representative process embodiments, wherein peptides are made using SPPS techniques, will now be described in more detail. Any type of support suitable in the practice of SPPS can be used in accordance with the inventive methods. In preferred embodiments, the support comprises a resin that can be made from one or more polymers, copolymers, or combinations of polymers such as polyamide, polysulfamide, substituted polyethylenes, polyethylene glycol, phenolic resins, polysaccharides, or polystyrene. The polymer support can also be any solid that is sufficiently insoluble and inert to solvents used in peptide synthesis. The solid support typically includes a linking moiety to which the growing peptide is coupled during synthesis and which can be cleaved under desired conditions to release the peptide from the support. Suitable solid supports can include linkers that are photocleavable, TFA-cleavable, HF-cleavable, fluoride ion-cleavable, reductively-cleavable, Pd(O)-cleavable, nucleophilically-cleavable, or radically-cleavable. Preferred linking moieties are cleavable under conditions such that the cleaved peptide is still substantially protected by side chain protecting groups.

Preferred solid supports include acid sensitive solid supports, for example, hydroxymethyl-polystyrene-divinylbenzene polymer resin (“Wang” resins, see Wang, S. S. 1973, J. Am. Chem. Soc., 95: 1328-33), 2-chlorotrityl chloride resin (see Barlos et al. (1989) Tetrahedron Letters 30(30): 3943-3946), and 4-hydroxymethyl-3-methoxyphenoxybutyric acid resin (see Richter et al. (1994), Tetrahedron Letters 35(27): 4705-4706), as well as functionalized, crosslinked poly N-acryloylpyrrolidone resins, and chloromethylpolystyrene dinvinylbenzene polymer resins. These types of solid supports are commercially available from, for example, Calbiochem-Novabiochem Corp., San Diego, Calif.

When SPPS is utilized, the synthesized peptide is preferably cleaved from the solid support (such as a resin) prior to utilization of the inventive methods described herein. Peptides synthesized via SPPS techniques can be cleaved using techniques well known to those skilled in the art. For example, solutions of 1% or 2% trifluoracetic acid (TFA) in DCM or a combination of a 1% and a 2% solution of TFA in DCM can be used to cleave the peptide. Alternatively, acetic acid (HOAC) can be used to cleave the peptide. The specific cleavage reagent, solvents and time selected for cleavage will depend upon the particular peptide being cleaved. These parameters are within the skill in the relevant art.

General procedures for production and loading of resins that can be utilized in SPPS are described in “Principles and Practice of Solid Phase Peptide Synthesis” (Edited by Greagory A. Grant, 1992, W.H. Freeman and Company) and references therein, and are well known to those of ordinary skill in the art. Specific procedures for loading of Wang resins are described for example in Sieber (1987) Tet. Lett. 28: 6147-50, and Granadas et al. (1989), Int. J. Pept. Protein Res. 33: 386-90.

As noted herein, Fmoc is a protecting group used in certain embodiments for protection of the alpha-amino moiety of an amino acid. Depending upon which amino acid is being loaded, and at what point in the peptide fragment intermediate it is to be attached, the side chain of the amino acid may or may not be protected.

In some embodiments, the peptide fragment intermediates of the invention are synthesized by SSPS techniques using standard Fmoc protocols. See, for example, Carpin et al. (1970), J. Am. Chem. Soc. 92(19): 5748-5749; Carpin et al. (1972), J. Org. Chem. 37(22): 3404-3409, “Fmoc Solid Phase Peptide Synthesis,” Weng C. Chan and Peter D. White Eds. (2000) Oxford University Press Oxford Eng. The Fmoc-protected amino acids, either with or without side-chain protecting groups as desired, that are used in loading the resin and in peptide synthesis are available commercially from Genzyme Pharmaceuticals Inc., Cambridge, Mass.; Bachem Biosciences Inc., Torrance, Calif.; Senn Chemicals, Dielsdorf, Switzerland; and Orpegen Pharma, Heidelberg, Germany, or are readily synthesized using materials and methods well known in the art. As an alternative to the above procedure, the resin can be purchased, for example, pre-loaded with the appropriate Fmoc-alpha-N-protected amino acid (for example, from Bachem Biosciences Inc. or Senn Chemicals).

The loaded resin is washed with a solvent, such as NMP. The resin is then agitated with nitrogen bubbling in a swelling solvent to swell the resin beads. The Fmoc group is removed from the terminal amine using piperidine in NMP. The deprotected resin is then washed with NMP to remove Fmoc by-products and residual piperidine.

The amino acid residue or fragment to be coupled is activated for reaction at its carboxy terminus and coupled. The coupling cycle is repeated for each of the subsequent amino acid residues of the peptide fragment intermediate. Following the final coupling cycle, the resin is washed with a solvent such as NMP, and then washed with an inert second solvent such as DCM. Peptide fragment intermediates synthesized via SPPS techniques can be cleaved from the resin using techniques well known to those of skill in the art, for example by the addition of a solution of an acid such as TFA in DCM. The cleaved peptide intermediate can then be isolated.

The present invention is directed to synthetic methods for making the peptide relaxin (RLX) Chain B using solid and/or solution phase techniques. Peptide molecules of the invention may be protected, unprotected, or partially protected. Protection may include N-terminus protection, side chain protection, and/or C-terminus protection. While the invention is generally directed at the synthesis of relaxin Chain B, its counterparts, fragments and their counterparts, and fusion products and their counterparts of these, the inventive teachings herein can also be applicable to the synthesis of other peptides, particularly those that are synthesized using a combination of solid phase and solution phase approaches. The invention is also applicable to the synthesis of peptide intermediate fragments associated with impurities, particularly pyroglutamate impurities. Preferred relaxin Chain B molecules useful in the practice of the present invention include natural and non-natural relaxin Chain B and counterparts thereof.

As used herein, a “counterpart” refers to natural and non-natural analogs, derivatives, fusion compounds, salts, or the like of a peptide. As used herein, a peptide analog generally refers to a peptide having a modified amino acid sequence such as by one or more amino acid substitutions, deletions, inversions, and/or additions relative to another peptide or peptide counterpart. Substitutions may involve one or more natural or non-natural amino acids. Substitutions preferably may be conservative or highly conservative. A conservative substitution refers to the substitution of an amino acid with another that has generally the same net electronic charge and generally the same size and shape. For instance, amino acids with aliphatic or substituted aliphatic amino acid side chains have approximately the same size when the total number of carbon and heteroatoms in their side chains differs by no more than about four. They have approximately the same shape when the number of branches in their side chains differs by no more than about one or two. Amino acids with phenyl or substituted phenyl groups in their side chains are considered to have about the same size and shape. Listed below are five groups of amino acids. Replacing an amino acid in a compound with another amino acid from the same groups generally results in a conservative substitution.

Group I: glycine, alanine, valine, leucine, isoleucine, serine, threonine, cysteine, methionine and non-naturally occurring amino acids with C₁-C₄ aliphatic or C₁-C₄ hydroxyl substituted aliphatic side chains (straight chained or monobranched).

Group II: glutamic acid, aspartic acid and normaturally occurring amino acids with carboxylic acid substituted C₁-C₄ aliphatic side chains (unbranched or one branch point).

Group III: lysine, ornithine, arginine and normaturally occurring amino acids with amine or guanidino substituted C₁-C₄ aliphatic side chains (unbranched or one branch point).

Group IV: glutamine, asparagine and non-naturally occurring amino acids with amide substituted C₁-C₄ aliphatic side chains (unbranched or one branch point).

Group V: phenylalanine, phenylglycine, tyrosine and tryptophan.

A “highly conservative substitution” is the replacement of an amino acid with another amino acid that has the same functional group in the side chain and nearly the same size and shape. Amino acids with aliphatic or substituted aliphatic amino acid side chains have nearly the same size when the total number carbon and heteroatoms in their side chains differs by no more than two. They have nearly the same shape when they have the same number of branches in their side chains. Examples of highly conservative substitutions include valine for leucine, threonine for serine, aspartic acid for glutamic acid and phenylglycine for phenylalanine.

A peptide derivative generally refers to a peptide, a peptide analog, or other peptide counterpart having chemical modification of one or more of its side groups, alpha carbon atoms, terminal amino group, and/or terminal carboxyl acid group. By way of example, a chemical modification includes, but is not limited to, adding chemical moieties, creating new bonds, and/or removing chemical moieties. Modifications at amino acid side groups include, without limitation, acylation of lysine e-amino groups, N-alkylation of arginine, histidine, or lysine, alkylation of glutamic or aspartic carboxylic acid groups, and deamidation of glutamine or asparagine. Modifications of the terminal amino group include, without limitation, the des-amino, N-lower alkyl, N-di-lower alkyl, and N-acyl (e.g., —CO-lower alkyl) modifications. Modifications of the terminal carboxy group include, without limitation, the amide, lower alkyl amide, dialkyl amide, and lower alkyl ester modifications. Thus, partially or wholly protected peptides constitute peptide derivatives.

In preferred embodiments, the present invention provides methodologies for synthesizing synthetic relaxin Chain B peptides having the following formula

(SEQ. ID NO. 7):
Z-Asp-Ser-Trp-Met-Glu-Glu-Val-Ile-Lys-Leu-Cys-Gly-
Arg-Glu-Leu-Val-Arg-Ala-Gln-Ile-Ala-Ile-Cys-Gly-
Met-Ser-Thr-Trp-Ser-OH

wherein:
Z is H or N-terminal protecting group Boc- or Fmoc-.

In a preferred embodiment (SEQ. ID NO. 7) has the formula:

H-Asp-Ser-Trp-Met-Glu-Glu-Val-Ile-Lys-Leu-Cys-Gly-
Arg-Glu-Leu-Val-Arg-Ala-Gln-Ile-Ala-Ile-Cys-Gly-
Met-Ser-Thr-Trp-Ser-OH

The present invention provides improved methodologies for making relaxin

Chain B peptides including side chain protected versions such as the peptide having the formula

(SEQ ID NO. 6):
Z-Asp-Ser-Trp-Met-Glu-Glu-Val-Ile-Lys-Leu-Cys-Gly-
Arg-Glu-Leu-Val-Arg-Ala-Gln-Ile-Ala-Ile-Cys-Gly-
Met-Ser-Thr-Trp-Ser-OtBu

wherein:
Z is H or N-terminal protecting group Boc- or Fmoc-; and
one or more residues of said sequence optionally include side chain protection.

In a preferred embodiment (SEQ. ID NO. 6) has the formula:

Boc-Asp(OtBu)-Ser(tBu)-Trp(Boc)-Met-Glu(OtBu)-Glu
(OtBu)-Val-Ile-Lys(Boc)-Leu-Cys(Trt)-Gly-Arg(Pbf)-
Glu(OtBu)-Leu-Val-Arg(Pbf)-Ala-Gln(Trt)-Ile-Ala-
Ile-Cys(Trt)-Gly-Met-Ser(OtBu)-Thr(OtBu)-Trp-Ser
(OtBu)-OtBu.

The present invention provides improved methodologies for making relaxin Chain B peptides including side chain protected versions of fragments thereof such as the peptide having the formula

(SEQ ID NO. 5):
Z-Arg-Glu-Leu-Val-Arg-Ala-Gln-Ile-Ala-Ile-Cys-Gly-
Met-Ser-Thr-Trp-Ser-OtBu

wherein:
Z is H or N-terminal protecting group Fmoc-; and
one or more residues of said sequence optionally include side chain protection.

In a preferred embodiment (SEQ. ID NO. 5) has the formula:

H-Arg(Pbf)-Glu(OtBu)-Leu-Val-Arg(Pbf)-Ala-
Gln(Trt)-Ile-Ala-Ile-Cys(Trt)-Gly-Met-Ser(OtBu)-
Thr(OtBu)-Trp-Ser(OtBu)-OtBu

In a preferred embodiment (SEQ. ID NO. 5) has the formula:

Fmoc-Arg(Pbf)-Glu(OtBu)-Leu-Val-Arg(Pbf)-Ala-
Gln(Trt)-Ile-Ala-Ile-Cys(Trt)-Gly-Met-Ser(OtBu)-
Thr(OtBu)-Trp-Ser(OtBu)-OtBu

The present invention provides improved methodologies for making relaxin Chain B peptides including side chain protected versions of fragments thereof such as the peptide having the formula

| | |
|---|---|
| (SEQ ID NO. 4): | Z-Met-Ser-Thr-Trp-Ser-OtBu |

wherein:
Z is H or N-terminal protecting group Fmoc-; and
one or more residues of said sequence optionally include side chain protection.

In a preferred embodiment (SEQ. ID NO. 4) has the formula:

H-Met-Ser(OtBu)-Thr(OtBu)-Trp-Ser(OtBu)-OtBu

In a preferred embodiment (SEQ. ID NO. 4) has the formula:

Fmoc-Met-Ser(OtBu)-Thr(OtBu)-Trp-Ser(OtBu)-OtBu

The present invention provides improved methodologies for making relaxin Chain B peptides including side chain protected versions of fragments thereof such as the peptide having the formula

| | |
|---|---|
| (SEQ ID NO. 3): | Z-Met-Ser-Thr-Trp-OH |

wherein:
Z is H- or N-terminal protecting group Fmoc; and
one or more residues of said sequence optionally include side chain protection.

In a preferred embodiment (SEQ. ID NO. 3) has the formula:

H-Met-Ser(OtBu)-Thr(OtBu)-Trp-OH

In a preferred embodiment (SEQ. ID NO. 3) has the formula:

Fmoc-Met-Ser(OtBu)-Thr(OtBu)-Trp-OH

The present invention provides improved methodologies for making relaxin Chain B peptides including side chain protected versions of fragments thereof such as the peptide having the formula

(SEQ ID NO. 2)

Z-Arg-Glu-Leu-Val-Arg-Ala-Gln-Ile-Ala-Ile-Cys-Gly-
OH

wherein:
Z is H or N-terminal protecting group Fmoc-; and
one or more residues of said sequence optionally include side chain protection.

In a preferred embodiment (SEQ. ID NO. 2) has the formula:

H-Arg(Pbf)-Glu(OtBu)-Leu-Val-Arg(Pbf)-Ala-
Gln(Trt)-Ile-Ala-Ile-Cys(Trt)-Gly-OH

In a preferred embodiment (SEQ. ID NO. 2) has the formula: Fmoc-Arg(Pbf)-Glu(OtBu)-Leu-Val-Arg(Pbf)-Ala-Gln(Trt)-Ile-Ala-Ile-Cys(Trt)-Gly-OH

The present invention provides improved methodologies for making relaxin Chain B peptides including side chain protected versions such as the peptide having the formula

(SEQ ID NO. 1)

Z-Asp-Ser-Trp-Met-Glu-Glu-Val-Ile-Lys-Leu-Cys-Gly-
OH

wherein:
Z is H, N-terminal protecting group Boc- or Fmoc-; and
one or more residues of said sequence optionally include side chain protection.

In a preferred embodiment (SEQ. ID NO. 1) has the formula:

Boc-Asp(OtBu)-Ser(tBu)-Trp(Boc)-Met-Glu(OtBu)-
Glu(OtBu)-Val-Ile-Lys(Boc)-Leu-Cys(Trt)-Gly-OH

Alkali metal halides such as LiBr facilitate the couplings of the above fragments 2 and 3′ as well as 1 and 2+3′ by increasing solubility of the fragments. In a preferred embodiment, LiBr is used in the solution phase couplings to increase solubility of the fragments. The concentration may be from approximately 5 to 20 equivalents LiBr to equivalent fragment.

By way of example, Scheme 1 shows an illustrative scheme for synthesizing relaxin Chain B peptides and their counterparts. Scheme 1 is believed to be particularly suitable for the scaled-up synthesis of relaxin Chain B peptides. Scaled-up procedures are typically performed to provide an amount of peptide useful for commercial distribution. For example the amount of peptide in a scaled-up procedure can be 500 g, or 1 kg per batch, and more typically tens of kg to hundreds of kg per batch or more. In preferred embodiments, the inventive methods can provide such improvements as reduction in processing (synthesis) time, improvements in the yield of products, improvements in product purity, and/or reduction in amount of reagents and starting materials required.

The synthesis shown in Scheme 1 below uses a combination of solid and solution phase techniques to prepare the peptide product.

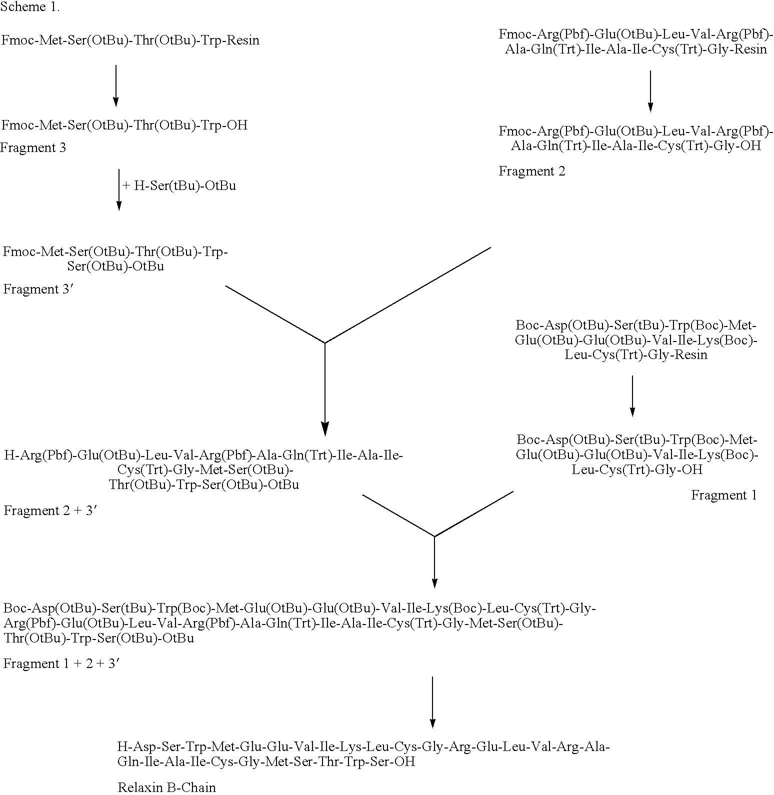

All patents, published patent applications, other publications, and pending patent applications cited in this specification are incorporated by reference herein in their respective entireties for all purposes.

### EXAMPLES

### Example 1

### Synthesis of Relaxin Chain B Fragment 2+3′ (H-AA(13-29)-OtBu)

The relaxin chain B Fragment 2 (Fmoc-AA(13-24)-OH) (10.88 g) and Fragment 3′ (H-AA(25-29)-OtBu) (4.44 g) was mixed in a 500 mL 3-neck round bottom flask with bath 25° C. The solution of LiBr (3.5 g) in THF (120 mL) and NMP (80 mL) was charged to the flask containing the mixture while stirring. The pot temperature spiked from 24° C. to 27° C. After 10 min agitation, HOBt Hydrate (0.64 g) and BOP (2.0 g) were charged to this solution with THF (20 mL) rinse. Then, DIEA (1.6 mL) was charged to the reaction mixture. The reaction was agitated at 25° C. bath and monitored by HPLC. Overnight reaction completion check indicated that the reaction was not completed. Two kicker charges were performed (first, 0.304 g of Fragment 2/0.099 g of BOP/0.16 mL of DIEA, and later, 0.605 g of Fragment 2/0.20 g of BOP/0.32 mL of DIEA). After the total of 20 hour reaction, the coupling was completed. Piperidine (5.0 mL) was charged to the reaction mixture. After 2 hours' stirring, the Fmoc removal was done. Then Pyridine Hydrochloride (10.01 g) was charged to the reaction mixture with a 2.5° C. temperature spike. After stirring for 35 min, the reaction mixture then was quenched in water (800 mL) with 15° C. bath. After 20 min stirring, the solid was isolated by filtering, washing with water (2×100 mL) and drying overnight to give 16.37 g solid. The isolated solid (15.4 g) then was re-slurried in a 250 mL mixture of MTBE/n-Heptane (50/50) at 35° C. for 1.5 hour, and at 25° C. for 3 hours. Then the solid was isolated by filtering, washing with n-Heptane (2×20 mL) and drying over weekend to give 14.66 g solid product with purity of 48.6% AN. Yield: 104.4% (based on Fragment 2).

### Example 2

### Synthesis of Relaxin Chain B Fragment 1+2+3′ (Boc-AA(1-29)-OtBu)

The relaxin chain B Fragment 2+3′ (H-AA(13-29)-OtBu) (13.46 g) and Fragment 1 Boc-AA(1-12)-OH) (6.07 g) was mixed in a 500 mL 3-neck round bottom flask with 25° C. bath. The solution of LiBr (3.5 g) in THF (120 mL) and NMP (80 mL) was charged to the flask containing the mixture while stirring. The reaction temperature spiked to 28° C. After 15 min agitation, HOBt Hydrate (0.65 g), BOP (2.0 g) and DIEA (1.6 mL) were charged to this solution with NMP (5 mL) rinse. The reaction was agitated at 25° C. bath and monitored by HPLC. Base on the HPLC results, two kicker charges were performed (first, 1.17 g of Fragment 1/0.44 g of BOP/0.3 mL of DIEA/5 mL THF rinse, and later, 1.50 g of Fragment 1/0.43 g of BOP/0.3 mL of DIEA/5 mL THF rinse). Overnight reaction completion check indicated that one more kicker charge was needed (0.2 g of BOP/0.2 mL of DIEA). After the total of 20 hour reaction, the coupling was completed. The reaction mixture then was quenched in water (800 mL) with 10° C. bath. After 60 min stirring at 25° C., the solid was isolated by filtering, washing with water (4×50 mL) and drying overnight to give 20.77 g solid product with purity of 56.4% AN. Yield: 93.2% (based on Fragment 2+3′)

### Example 3

### Synthesis of Relaxin Chain B (H-AA(1-29)-OH) Crude

The slurry of the relaxin chain B Fragment 1+2+3′(Boc-AA(1-29)-OtBu) (20.25 g) in DCM (80 mL) was mixed with the deprotection cocktail containing TFA (293 mL), DTT (13 g) and Water (13 mL) at 25° C. bath. After 3 hours' agitation, the reaction mixture was cooled to −5° C., and quenched by charging cold (−20° C.) MTBE (1200 mL) to it in 15 min while maintaining the reaction temperature <18° C. The quenched reaction mixture was stirred at 15° C. bath for 45 min. The solid product was filtered, washed with MTBE (5×50 mL), and dried overnight in vacuum oven. A 15.68 g of relaxin Chain B Crude (21.8% wt/wt) was obtained with a purity of 19.4% AN. Yield: 127.4% (based on Fragment 1+2+3′).

### Synthesis of Relaxin Chain B, H-AA(1-29)-OH

H-Asp-Ser-Trp-Met-Glu-Glu-Val-Ile-Lys-Leu-Cys-Gly-Arg-Glu-Leu-Val-Arg-Ala-Gln-Ile-Ala-Ile-Cys-Gly-Met-Ser-Thr-Trp-Ser-OH

### Example 4

### Synthesis of Relaxin Chain B Fragment 1, Boc-Asp(OtBu)-Ser(tBu)-Trp(Boc)-Met-Glu(OtBu)-Glu(OtBu)-Val-Ile-Lys(Boc)-Leu-Cys(Trt)-Gly-OH

The solid phase synthesis of Fmoc-AA(1-12)-O-2CT resin was carried out in a 3-L, glass-fritted resin flask. H-Gly-2-CT resin (100.00 g) with a loading of 0.43 mmol/g was charged to the resin flask and swelled in DCM (1000 mL) for 30 min at ambient temperature. The dichloromethane (DCM) solvent was drained, and the resin was washed with N-methyl-2-pyrrolidinone (NMP) (3×1000 mL).

All Fmoc deprotections of the resin were carried out by treating the resin with 20% piperidine/40% NMP/40% DMSO (v/v/v) (2×1000 mL). After the second piperidine/NMP/DMSO treatment, the resin was sequentially washed with NMP (1000 mL), IPA (2×1000 mL), DCM (1000 mL), IPA (2×1000 mL), NMP (1000 mL), IPA (2×1000 mL), and 50% NMP/50% DMSO (3×1000 mL).

To prepare the activated ester solution, the amino acid and 6-chloro-hydroxybenzotriazole hydrate (6-Cl HOBT) were weighed, dissolved in NMP, treated with diisopropylcarbodiimide (DIC), and diluted with DMSO in a flask. The resultant solution was added to resin flask, the preparation flask was rinsed with DMSO into the resin flask, which was then stirred with the resin for 190-295 min at ambient temperature. A sample was taken for a bromophenol blue (BPB) test to confirm reaction completion. After the coupling reaction was complete, the coupling solution was drained and the resin was sequentially washed with NMP (1000 mL), IPA (2×1000 mL), and 50% NMP/50% DMSO (v/v) (2×1000 mL).

The sequence of removing the Fmoc group and coupling the next amino acid was repeated for the remaining amino acids in the fragment (i.e., in the order of Cys(Trt)→Leu→Lys(Boc)→Ile→Val→Glu(OtBu)→Glu(OtBu)→Met→Trp(Boc)→Ser(tBu)→Boc-Asp(OtBu).

The fully-built peptide was cleaved from the resin by stirring the resin in 50% TFE (trifluoroethanol)/50% DCM (v/v) (1000 mL) at ambient temperature for 15.5 h. The cleavage solution was drained, and the resin was washed with DCM (6×1000 mL). Removal of solvents under vacuum from the combined filtrates at 20° C.-25° C. gave a 98.4% yield of Chain B Fragment 1 (78.8% purity, AN).

All reagent amounts used in this example are listed in the following table:

| | Amino | 6-Cl | | DIC | | | Coupling |
|---|---|---|---|---|---|---|---|
| | Acid | HOBT | NMP | (mL)/ | DMSO | Piperidine | Time |
| Material | (g)/Eq | (g)/Eq | (mL) | Eq | (mL) | (mL) | (min) |
| Fmoc- | 50.37/2.00 | 21.88/3.00 | 500 | 20.0/3.00 | 500 | 400 | 197 |
| Cys(Trt)- | | | | | | | |
| OH | | | | | | | |
| Fmoc- | 30.39/2.00 | 21.88/3.00 | 500 | 20.0/3.00 | 500 | 400 | 229 |
| Leu-OH | | | | | | | |
| Fmoc- | 40.29/2.00 | 21.88/3.00 | 500 | 20.0/3.00 | 500 | 400 | 295 |
| Lys(Boc)- | | | | | | | |
| OH | | | | | | | |
| Fmoc-Ile- | 30.39/2.00 | 21.88/3.00 | 500 | 20.0/3.00 | 500 | 400 | 197 |
| OH | | | | | | | |
| Fmoc- | 29.19/2.00 | 21.88/3.00 | 500 | 20.0/3.00 | 500 | 400 | 236 |
| Val-OH | | | | | | | |
| Fmoc- | 38.14/2.00 | 21.88/3.00 | 500 | 33.3/5.00 | 500 | 400 | 198 |
| Glu(OtBu)- | | | | | | | |
| OH•H₂O | | | | | | | |
| Fmoc- | 38.14/2.00 | 21.88/3.00 | 500 | 33.3/5.00 | 500 | 400 | 204 |
| Glu(OtBu)- | | | | | | | |
| OH•H₂O | | | | | | | |
| Fmoc- | 31.94/2.00 | 21.88/3.00 | 500 | 20.0/3.00 | 500 | 400 | 197 |
| Met-OH | | | | | | | |
| Fmoc- | 45.29/2.00 | 21.88/3.00 | 500 | 20.0/3.00 | 500 | 400 | 190 |
| Trp(Boc)- | | | | | | | |
| OH | | | | | | | |
| Fmoc- | 32.98/2.00 | 21.88/3.00 | 500 | 20.0/3.00 | 500 | 400 | 206 |
| Ser(tBu)- | | | | | | | |
| OH | | | | | | | |
| Boc- | 24.88/2.00 | 21.88/3.00 | 500 | 20.0/3.00 | 500 | 400 | 198 |
| Asp(OtBu)- | | | | | | | |
| OH | | | | | | | |

### Example 5

### Synthesis of Relaxin Chain B Fragment 2; Fmoc-AA(13-24)-OH; Fmoc-Arg(Pbf)-Glu(OtBu)-Leu-Val-Arg(Pbf)-Ala-Gln(Trt)-Ile-Ala-Ile-Cys(Trt)-Gly-OH

The solid phase synthesis of Fmoc-AA(13-24)-O-2CT resin was carried out in a 1.5-L, glass-fritted resin flask. H-Gly-2-CT resin (85.00 g) with a loading of 0.43 mmol/g was charged to the resin flask and swelled in DCM (850 mL) for 30 min at ambient temperature. The DCM solvent was drained, and the resin was washed with NMP (3×510 mL).

All Fmoc deprotections of the resin were carried out by treating the resin with 20% piperidine/80% NMP (v/v) (2×595 mL) for 30 min each. After the second piperidine/NMP treatment, the resin was sequentially washed with NMP (3×850 mL), DCM (2×850 mL), and NMP (2×850 mL).

To prepare the activated ester solution, the amino acid and 6-Cl HOBT were weighed, dissolved in NMP, treated with DIC, and diluted with DMSO in a flask. The resultant solution was added to resin flask. The preparation flask was rinsed with DMSO into the resin flask, which was then stirred with the resin for 3-67 hr at ambient temperature. A sample was taken for a Kaiser test to confirm reaction completion. After the coupling reaction was completed, the coupling solution was drained and the resin was washed with NMP (3×850 mL). The sequence of removing the Fmoc group and coupling the next amino acid was repeated for the remaining amino acids in the fragment (i.e., in the order of Cys(Trt)→Ile→Ala→Ile→Gln(Trt)→Ala→Arg(Pbf)→Val→Leu→Glu(OtBu)→Fmoc-Arg(Pbf).

Most of the fully-built peptide was cleaved from the resin by stirring the resin in 50% TFE/50% DCM (v/v) (850 mL) at ambient temperature for 18 h. The cleavage solution was drained, and the resin was washed with DCM (4×850 mL). The solvents were removed from the combined filtrates under vacuum at 20° C.-25° C. To remove the final traces of TFE, the product was slurred in DCM (910 mL) and toluene (200 mL) and stripped to a low volume on a rotary evaporator. Toluene was added and the slurry was stripped to a low volume. The product was dried in a vacuum oven at 11 mmHg at ambient temperature and gave an 81.0% yield of Chain B Fragment 2 (91.9% purity, a. n.).

All reagent amounts used in this example are listed in the following table:

| | Amino | 6-Cl | | DIC | | | Coupling |
|---|---|---|---|---|---|---|---|
| | Acid | HOBT | NMP | (mL)/ | DMSO | Piperidine | Time |
| Material | (g)/Eq | (g)/Eq | (mL) | Eq | (mL) | (mL) | (hr) |
| Fmoc- | 42.81/2.00 | 18.60/3.00 | 425 | 17.0/3.00 | 425 | 238 | 18 |
| Cys(Trt)- | | | | | | | |
| OH | | | | | | | |
| Fmoc-Ile- | 25.83/2.00 | 18.60/3.00 | 425 | 17.0/3.00 | 425 | 238 | 3 |
| OH | | | | | | | |
| Fmoc- | 24.07/2.00 | 18.60/3.00 | 425 | 17.0/3.00 | 425 | 238 | 44 |
| Ala- | | | | | | | |
| OH•H₂O | | | | | | | |
| Fmoc-Ile- | 25.83/2.00 | 18.60/3.00 | 425 | 17.0/3.00 | 425 | 238 | 67 |
| OH | | | | | | | |
| Fmoc- | 44.64/2.00 | 18.60/3.00 | 425 | 17.0/3.00 | 425 | 238 | 3 |
| Gln(Trt)- | | | | | | | |
| OH | | | | | | | |
| Fmoc- | 24.07/2.00 | 18.60/3.00 | 425 | 17.0/3.00 | 425 | 238 | 15 |
| Ala- | | | | | | | |
| OH•H₂O | | | | | | | |
| Fmoc- | 47.43/2.00 | 18.60/3.00 | 425 | 17.0/3.00 | 425 | 238 | 3 |
| Arg(Pbf)- | | | | | | | |
| OH | | | | | | | |
| Fmoc- | 24.81/2.00 | 18.60/3.00 | 425 | 17.0/3.00 | 425 | 238 | 16 |
| Val-OH | | | | | | | |
| Fmoc- | 25.83/2.00 | 18.60/3.00 | 425 | 17.0/3.00 | 425 | 238 | 3 |
| Leu-OH | | | | | | | |
| Fmoc- | 31.10/2.00 | 18.60/3.00 | 425 | 17.0/3.00 | 425 | 238 | 15 |
| Glu(OtBu)- | | | | | | | |
| OH | | | | | | | |
| Fmoc- | 47.43/2.00 | 18.60/3.00 | 425 | 17.0/3.00 | 425 | 238 | 4.5 |
| Arg(Pbf)- | | | | | | | |
| OH | | | | | | | |

### Example 6

### Synthesis of Relaxin Chain B Fragment 3; Fmoc-AA(25-28)-OH; Fmoc-Met-Ser(OtBu)-Thr(OtBu)-Trp-OH

The solid phase synthesis of Fmoc-AA(25-28)-O-2CT resin was carried out in a 1.5-L, glass-fritted resin flask. H-Trp-2-CT resin (100.00 g) with a loading of 0.65 mmol/g was charged to the resin flask and swelled in DCM (1000 mL) for 30 min at ambient temperature. The DCM solvent was drained, and the resin was washed with NMP (3×1000 mL).

All Fmoc deprotections of the resin were carried out by treating the resin with 20% piperidine/80% NMP (v/v) (2×600 mL) for 20 min each. After the second piperidine/NMP treatment, the resin was washed with NMP (6×1000 mL).

To prepare the activated ester solution, the amino acid and 6-Cl HOBT were weighed, dissolved in NMP, treated with DIC, and diluted with DMSO in a flask. The resultant solution was added to resin flask. The preparation flask was rinsed with DMSO into the resin flask, which was then stirred with the resin for 2.5-7 hr at ambient temperature. A sample was taken for Kaiser and BPB tests to confirm reaction completion. After the coupling reaction was completed, the coupling solution was drained and the resin was washed with NMP (3×1000 mL). The sequence of removing the Fmoc group and coupling the next amino acid was repeated for the remaining amino acids in the fragment (i.e., in the order of Thr(OtBu)→Ser(OtBu)→Fmoc-Met-OH.

The fully-built peptide was cleaved from the resin by stirring the resin in 50% TFE/50% DCM (v/v) (1500 mL) at ambient temperature for 23.5 h. The cleavage solution was drained, and the resin was washed with DCM (4×1000 mL). The solvents were removed from the filtrate on a rotary evaporator under vacuum at 20° C.-25° C. The DCM washes were added sequentially to the distillation vessel after the previous strip was completed. The product was dried overnight at 19 mmHg at ambient temperature and gave a 104.0% yield of Chain B Fragment 3 (95.2% purity, a. n.).

All reagent amounts used in this example are listed in the following table:

| | Amino | 6-Cl | | | | | Coupling |
|---|---|---|---|---|---|---|---|
| | Acid | HOBT | NMP | DIC | DMSO | Piperidine | Time |
| Material | (g)/Eq | (g)/Eq | (mL) | (mL)/Eq | (mL) | (mL) | (hr) |
| Fmoc- | 51.57/2.00 | 33.07/3.00 | 520 | 30.2/3.00 | 260 | 400 | 7 |
| Thr(OtBu)- | | | | | | | |
| OH | | | | | | | |
| Fmoc- | 49.85/2.00 | 33.07/3.00 | 520 | 30.2/3.00 | 260 | 400 | 3 |
| Ser(OtBu)- | | | | | | | |
| OH | | | | | | | |
| Fmoc- | 48.29/2.00 | 33.07/3.00 | 520 | 30.2/3.00 | 260 | 400 | 2.5 |
| Met-OH | | | | | | | |

### Example 7

### Synthesis of Relaxin Chain B Fmoc-Fragment 3′; Fmoc-AA(25-29)-OtBu; Fmoc-Met-Ser(OtBu)-Thr(OtBu)-Trp-Ser(OtBu)-OtBu

The relaxin Chain B Fragment Fmoc-3 (62.09 g, 64.80 mmol) was dissolved in DCM (700 mL). H-Ser(tBu)-OtBu (28.34 g, 130.4 mmol, 2.0 equiv.), N-hydroxysuccinimide (29.83 g, 259.2 mmol, 4.0 equiv.), and 4-methylmorpholine (29.2 mL, 265.5 mmol, 4.1 equiv.) were added sequentially. The reaction solution was cooled to 0° C., and N-(3-dimethylaminopropyl)-N-ethylcarbodiimide hydrochloride (EDAC.HCl) (49.90 g, 260.3 mmol, 4.0 equiv.) was added. After 64 hr, the ratio of Fmoc 3/Fmoc 3′ was 20.4/67.2 by HPLC. Kicker charges of all the reagents were added, 1 equiv. of N-hydroxysuccinimide, 4-methylmorpholine, and EDAC.HCl and 0.5 equiv. of H-Ser(tBu)-OtBu. The reaction was stirred another 15 hr at 0° C. After the kicker charges the Fmoc 3/Fmoc 3′ ratio was 1.4/85.8, indicating the reaction was completed. The solution was extracted with 8% NaHCO₃ (3×310 mL). The organic layer was washed with 50% citric acid (300 mL). The solvent was stripped to a low volume, and replaced with TFE. The product was precipitated from solution by adding deionized (DI) water (1500 mL), filtered, washed with DI water (2500 mL), and dried over night at ambient temperature. A weight of 81.04 g (108.0%) of Chain B Fmoc-Fragment 3′ (Fmoc-AA(25-29)-OtBu) was obtained with a purity of 87.3% a. n. by HPLC.

### Example 8

### Synthesis of Relaxin Chain B Fragment 3′; H AA(25-29)-OtBu; H-Met-Ser(OtBu)-Thr(OtBu)-Trp-Ser(OtBu)-OtBu

The relaxin Chain B Fragment Fmoc-3′ (48.01 g, 41.47 mmol) and diethylamine (25 mL, 17.68 g, 242 mmol) were dissolved in dimethylformamide (DMF) (125 mL) and stirred at room temperature for 4 hr. The reaction solution was cooled to 10-12° C. and heptane (3000 mL) was added. The product oiled out in the DMF layer, so the heptane was decanted. The Fragment 3′ was precipitated by adding heptane (5500 mL) at 18° C., and stirring for 30 min. The product was filtered and dried over night at ambient temperature, yielding 32.7 g (84.3%) of crude 3′ which had an HPLC purity of 85.7% a. n. This crude produce was redissolved in THF (250 mL) and precipitated with DI water (3000 mL), which was filtered, washed with DI water (1000 mL) and dried at ambient temperature over the weekend, yielding 29.08 g (75.0% from Fmoc-fragment 3′) with a purity of 87.1% a. n. by HPLC.

### Example 9

### Synthesis of Relaxin Chain B Fragment 2+3′; H-AA(13-29)-OtBu; H-Arg(Pbf)-Glu(OtBu)-Leu-Val-Arg(Pbf)-Ala-Gln(Trt)-Ile-Ala-Ile-Cys(Trt)-Gly-Met-Ser(OtBu)-Thr(OtBu)-Trp-Ser(OtBu)-OtBu

The relaxin Chain B Fragment 3′ (43.83 g, 46.87 mmol) and Fragment 2 (117.48 g, 45.25 mmol) were placed in a reaction flask. The mixture was stirred slowly. Lithium bromide (LiBr) (35.00 g, 403.0 mmol) and THF (1200 mL) were dissolved in NMP (800 mL). This solution was added to the slowly stirring peptides at 22° C. and stirred until the peptides dissolved. (Benzotriazol-1-yloxy)tris(dimethylamino) phosphonium hexafluorophosphate (BOP reagent) (23.42 g, 52.95 mmol), 1-hydroxybenzotriazole hydrate (HOBT.H₂O) (6.93 g, 51.29 mmol), diisopropylethylamine (DIEA) (19.8 mL, 14.69 g, 113.7 mmol) and THF (110 mL) were added and the solution was stirred at ambient temperature overnight. Piperidine (50 mL) was added and stirred for 2 hr. Pyridine hydrochloride (125.53 g, 10.9 mol) was added and stirred for 15 min. The reaction solution was added to rapidly stirring cool (10-15° C.) DI water (8100 mL) over ˜3 min, stirred for another 20 min, filtered, washed with DI water, and dried at ambient temperature overnight, yielding 153.70 g, (95.3%) of Fragment 2,3′.

### Example 10

### Synthesis of Relaxin Chain B Fragment 1+2+3′: Boc-AA(1-29)-OtBu; Boc-Asp(OtBu)-Ser(tBu)-Trp(Boc)-Met-Glu(OtBu)-Glu(OtBu)-Val-Ile-Lys(Boc)-Leu-Cys(Trt)-Gly-Arg(Pbf)-Glu(OtBu)-Leu-Val-Arg(Pbf)-Ala-Gln(Trt)-Ile-Ala-Ile-Cys(Trt)-Gly-Met-Ser(OtBu)-Thr(OtBu)-Trp-Ser(OtBu)-OtBu

The relaxin Chain B Fragment 2+3′ (131.22 g) and Fragment 1 (86.92 g) were placed in a reaction flask. The mixture was stirred slowly. Lithium bromide (35.00 g, 403.0 mmol) and THF (1200 mL) were dissolved in NMP (800 mL). This solution was added to the slowly stirring peptides at 22° C. and stirred until the peptides dissolved. BOP reagent (24.88 g, 56.25 mmol), HOBT.H₂O (7.03 g, 52.02 mmol), and DIEA (24.5 mL, 18.18 g, 140.7 mmol) were added and stirred at ambient temperature overnight. The reaction completion was followed by HPLC. Kicker charges (each containing ˜5% BOP reagent and ˜5% DIEA) were added until the reaction was completed. The reaction solution was added to rapidly stirring DI water (8100 mL), stirred for 1.5 hr, filtered, washed with DI water, and dried at ambient temperature, yielding 216.72 g, (99.3%) of Boc-Fragment 1, 2, 3′.

### Example 11

### Synthesis of Relaxin Chain B; H-AA(1-29)-OH; H-Asp-Ser-Trp-Met-Glu-Glu-Val-Ile-Lys-Leu-Cys-Gly-Arg-Glu-Leu-Val-Arg-Ala-Gln-Ile-Ala-Ile-Cys-Gly-Met-Ser-Thr-Trp-Ser-OH

A solution of trifluoroacetic acid (TFA) (2880 mL), DCM (360 mL), dithiothreitol (DTT) (181.0 g, 1.17 mol), and DI water (180 mL) was prepared and added to the relaxin Chain B Boc-Fragment 1, 2, 3′ (213.12 g (39.11 mmol). The reaction mixture was stirred at room temperature for 5 hr. The reaction was cooled to −5 to −10° C. and t-butyl methyl ether (MTBE) (12.0 L) was added slowly over 75 min, while maintaining the pot temperature at −5 to −10° C. The slurry was filtered, washed with MTBE and dried at ambient temperature, yielding 148.38 g (44.79 mmol, 114.5%) of relaxin Chain B.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

What is claimed is:

1. A process for preparing a relaxin Chain B peptide comprising the step of:
a) introducing a peptide fragment including the amino acid sequence of
(SEQ ID NO. 3)
Z-Met-Ser-Thr-Trp-OH
wherein:
Z is H—; and
one or more residues of said sequence optionally include side chain protection.

2. The process of claim 1, further comprising the steps of:
b) coupling the peptide fragment of step a) in solution to H-Ser-OtBu in order to provide a peptide fragment including the amino acid sequence of
(SEQ ID NO. 4)
Z-Met-Ser-Thr-Trp-Ser-OtBu
wherein:
Z is N-terminal protecting group Fmoc-; and
one or more residues of said sequence optionally include side chain protection; and
c) removing the N-terminal protecting group to afford a peptide fragment including the amino acid sequence of
(SEQ ID NO. 4)
Z-Met-Ser-Thr-Trp-Ser-OtBu
wherein:
Z is H; and
one or more residues of said sequence optionally include side chain protection.

3. The process of claim 2, further comprising the steps of:
d) introducing a peptide fragment including the amino acid sequence of
(SEQ ID NO. 2)
Z-Arg-Glu-Leu-Val-Arg-Ala-Gln-Ile-Ala-Ile-Cys-Gly-
OH
wherein:
Z is N-terminal protecting group Fmoc-; and
one or more residues of said sequence optionally include side chain protection;
e) coupling the peptide fragment of step d) in solution to the peptide fragment of step c) in the presence of an alkali metal halide in order to provide a peptide fragment including the amino acid sequence of
(SEQ ID NO. 5)
Z-Arg-Glu-Leu-Val-Arg-Ala-Gln-Ile-Ala-Ile-Cys-Gly-
Met-Ser-Thr-Trp-Ser-OtBu
wherein:
Z is N-terminal protecting group Fmoc-; and
one or more residues of said sequence optionally include side chain protection; and
f) removing the N-terminal protecting group to afford a peptide fragment including the amino acid sequence of
(SEQ ID NO. 5)
Z-Arg-Glu-Leu-Val-Arg-Ala-Gln-Ile-Ala-Ile-Cys-Gly-
Met-Ser-Thr-Trp-Ser-OtBu
wherein:
Z is H; and
one or more residues of said sequence optionally include side chain protection.

4. The process of claim 3, further comprising the steps of:
g) introducing a peptide fragment including the amino acid sequence of
(SEQ ID NO. 1)
Z-Asp-Ser-Trp-Met-Glu-Glu-Val-Ile-Lys-Leu-Cys-Gly-
OH
wherein:
Z is N-terminal protecting group Boc-; and
one or more residues of said sequence optionally include side chain protection; and
h) coupling the peptide fragment of step f) in solution to the peptide fragment of step g) in the presence of an alkali metal halide in order to provide a peptide including the amino acid sequence of
(SEQ ID NO. 6)
Z-Asp-Ser-Trp-Met-Glu-Glu-Val-Ile-Lys-Leu-Cys-Gly-
Arg-Glu-Leu-Val-Arg-Ala-Gln-Ile-Ala-Ile-Cys-Gly-
Met-Ser-Thr-Trp-Ser-OtBu
wherein:
Z is N-terminal protecting group Boc-; and
one or more residues of said sequence optionally include side chain protection.

5. The process of claim 4, further comprising the step of:
i) contacting the peptide resulting from step h) with acid in order to remove the N-terminal protecting group and deprotect the amino acid side chains to afford the deprotected relaxin Chain B amino acid sequence of
(SEQ ID NO. 7)
Z-Asp-Ser-Trp-Met-Glu-Glu-Val-Ile-Lys-Leu-Cys-Gly-
Arg-Glu-Leu-Val-Arg-Ala-Gln-Ile-Ala-Ile-Cys-Gly-
Met-Ser-Thr-Trp-Ser-OH
wherein:
Z is H.

6. A process for preparing a relaxin Chain B peptide comprising the step of:
a) introducing a peptide fragment including the amino acid sequence of
(SEQ ID NO. 3)
Z-Met-Ser(OtBu)-Thr(OtBu)-Trp-OH
wherein:
Z is N-terminal protecting group Fmoc-.

7. The process of claim 6, further comprising the step of:
b) coupling the peptide fragment of step a) in solution to H-Ser(tBu)-OtBu in order to provide a peptide fragment including the amino acid sequence of
(SEQ ID NO. 4)
Z-Met-Ser(OtBu)-Thr(OtBu)-Trp-Ser(OtBu)-OtBu
wherein:
Z is N-terminal protecting group Fmoc-.

8. The process of claim 7, further comprising the steps of:
c) removing the N-terminal protecting group to afford a peptide fragment including the amino acid sequence of
(SEQ ID NO. 4)
Z-Met-Ser(OtBu)-Thr(OtBu)-Trp-Ser(OtBu)-OtBu
wherein:
Z is H;
d) introducing a peptide fragment including the amino acid sequence of
(SEQ ID NO. 2)
Z-Arg(Pbf)-Glu(OtBu)-Leu-Val-Arg(Pbf)-Ala-Gln
(Trt)-Ile-Ala-Ile-Cys(Trt)-Gly-OH
wherein:
Z is N-terminal protecting group Fmoc-; and
e) coupling the peptide fragment of step c) in solution to the fragment of step d) in the presence of an alkali metal halide in order to provide a peptide fragment including the amino acid sequence of
(SEQ ID NO. 5)
Z-Arg(Pbf)-Glu(OtBu)-Leu-Val-Arg(Pbf)-Ala-Gln
(Trt)-Ile-Ala-Ile-Cys(Trt)-Gly-Met-Ser(OtBu)-Thr
(OtBu)-Trp-Ser(OtBu)-OtBu
wherein:
Z is N-terminal protecting group Fmoc-; and
f) removing the N-terminal protecting group to afford a peptide fragment including the amino acid sequence of
(SEQ ID NO. 5)
Z-Arg(Pbf)-Glu(OtBu)-Leu-Val-Arg(Pbf)-Ala-Gln
(Trt)-Ile-Ala-Ile-Cys(Trt)-Gly-Met-Ser(OtBu)-Thr
(OtBu)-Trp-Ser(OtBu)-OtBu
wherein:
Z is H.

9. The process of claim 8, further comprising the steps of:
g) introducing a peptide fragment including the amino acid sequence of
(SEQ. ID NO. 1)
Z-Asp(OtBu)-Ser(tBu)-Trp(Boc)-Met-Glu(OtBu)-Glu
(OtBu)-Val-Ile-Lys(Boc)-Leu-Cys(Trt)-Gly-OH
wherein:
Z is N-terminal protecting group Boc-;
h) coupling the peptide fragment of step f) in solution to the peptide fragment of step g) in the presence of an alkali metal halide in order to provide a peptide including the amino acid sequence of
(SEQ ID NO. 6)
Z-Asp(OtBu)-Ser(tBu)-Trp(Boc)-Met-Glu(OtBu)-Glu
(OtBu)-Val-Ile-Lys(Boc)-Leu-Cys(Trt)-Gly-Arg(Pbf)-
Glu(OtBu)-Leu-Val-Arg(Pbf)-Ala-Gln(Trt)-Ile-Ala-
Ile-Cys(Trt)-Gly-Met-Ser(OtBu)-Thr(OtBu)-Trp-Ser
(OtBu)-OtBu
wherein:
Z is N-terminal protecting group Boc-;
i) contacting the peptide resulting from step h) with acid in order to remove the N-terminal protecting group and deprotect the amino acid side chains to afford the deprotected relaxin Chain B amino acid sequence of
(SEQ ID NO. 7)
Z-Asp-Ser-Trp-Met-Glu-Glu-Val-Ile-Lys-Leu-Cys-Gly-
Arg-Glu-Leu-Val-Arg-Ala-Gln-Ile-Ala-Ile-Cys-Gly-
Met-Ser-Thr-Trn-Ser-OH
wherein:
Z is H.

10. A process for preparing a relaxin Chain B peptide comprising the step of:
a) introducing a peptide fragment including the amino acid sequence of
(SEQ ID NO. 2)
Fmoc-Arg(Pbf)-Glu(OtBu)-Leu-Val-Arg(Pbf)-Ala-
Gln(Trt)-Ile-Ala-Ile-Cys(Trt)-Gly-OH
wherein:
Z is N-terminal protecting group Fmoc-;
b) introducing a peptide fragment including the amino acid sequence of
(SEQ ID NO. 4)
H-Met-Ser(OtBu)-Thr(OtBu)-Trp-Ser(OtBu)-OtBu
wherein:
Z is H;
c) coupling the peptide fragment of step a) in solution to the fragment of step b) in the presence of an alkali metal halide in order to provide a peptide fragment including the amino acid sequence of
(SEQ ID NO. 5)
Z-Arg(Pbf)-Glu(OtBu)-Leu-Val-Arg(Pbf)-Ala-
Gln(Trt)-Ile-Ala-Ile-Cys(Trt)-Gly-Met-Ser(OtBu)-
Thr(OtBu)-Trp-Ser(OtBu)-OtBu
wherein:
Z is N-terminal protecting group Fmoc-;
d) removing the N-terminal protecting group to afford a peptide fragment including the amino acid sequence of
(SEQ ID NO. 5)
Z-Arg(Pbf)-Glu(OtBu)-Leu-Val-Arg(Pbf)-Ala-
Gln(Trt)-Ile-Ala-Ile-Cys(Trt)-Gly-Met-Ser(OtBu)-
Thr(OtBu)-Trp-Ser(OtBu)-OtBu
wherein:
Z is H.

11. A process for preparing a relaxin Chain B peptide comprising the step of:
a) introducing a peptide fragment including the amino acid sequence of
(SEQ ID NO. 5)
Z-Arg(Pbf)-Glu(OtBu)-Leu-Val-Arg(Pbf)-Ala-
Gln(Trt)-Ile-Ala-Ile-Cys(Trt)-Gly-Met-Ser(OtBu)-
Thr(OtBu)-Trp-Ser(OtBu)-OtBu
wherein:
Z is H;
b) introducing a peptide fragment including the amino acid sequence of
(SEQ. ID NO. 1)
Z-Asp(OtBu)-Ser(tBu)-Trp(Boc)-Met-Glu(OtBu)-
Glu(OtBu)-Val-Ile-Lys(Boc)-Leu-Cys(Trt)-Gly-OH
wherein:
Z is N-terminal protecting group Boc-;
c) coupling the peptide fragment of step a) in solution to the peptide fragment of step b) in the presence of an alkali metal halide in order to provide a peptide including the amino acid sequence of
(SEQ ID NO. 6)
Z-Asp(OtBu)-Ser(tBu)-Trp(Boc)-Met-Glu(OtBu)-
Glu(OtBu)-Val-Ile-Lys(Boc)-Leu-Cys(Trt)-Gly-
Arg(Pbf)-Glu(OtBu)-Leu-Val-Arg(Pbf)-Ala-Gln(Trt)-
Ile-Ala-Ile-Cys(Trt)-Gly-Met-Ser(OtBu)-Thr(OtBu)-
Trp-Ser(OtBu)-OtBu
wherein:
Z is N-terminal protecting group Boc.

12. A peptide of the amino acid sequence
(SEQ. ID NO. 1)
Z-Asp(OtBu)-Ser(tBu)-Trp(Boc)-Met-Glu(OtBu)-
Glu(OtBu)-Val-Ile-Lys(Boc)-Leu-Cys(Trt)-Gly-OH
wherein:
Z is H or N-terminal protecting group Boc.

13. A peptide of the amino acid sequence
(SEQ. ID NO. 2)
Z-Arg(Pbf)-Glu(OtBu)-Leu-Val-Arg(Pbf)-Ala-
Gln(Trt)-Ile-Ala-Ile-Cys(Trt)-Gly-OH
wherein:
Z is H or N-terminal protecting group Fmoc.

14. A peptide of the amino acid sequence
(SEQ. ID NO. 3)
Z-Met-Ser(OtBu)-Thr(OtBu)-Trp-OH
wherein:
Z is H or N-terminal protecting group Fmoc.

15. A peptide prepared by the process of claim 4 comprising the amino acid sequence
(SEQ ID NO. 4)
Z-Met-Ser(OtBu)-Thr(OtBu)-Trp-Ser(OtBu)-OtBu
wherein:
Z is H or N-terminal protecting group Fmoc.

16. A peptide of the amino acid sequence
(SEQ ID NO. 5)
Z-Arg(Pbf)-Glu(OtBu)-Leu-Val-Arg(Pbf)-Ala-
Gln(Trt)-Ile-Ala-Ile-Cys(Trt)-Gly-Met-Ser(OtBu)-
Thr(OtBu)-Trp-Ser(OtBu)-OtBu
wherein:
Z is H or N-terminal protecting group Fmoc.

17. A peptide of the amino acid sequence
(SEQ. ID NO. 6)
Z-Asp(OtBu)-Ser(tBu)-Trp(Boc)-Met-Glu(OtBu)-
Glu(OtBu)-Val-Ile-Lys(Boc)-Leu-Cys(Trt)-Gly-
Arg(Pbf)-Glu(OtBu)-Leu-Val-Arg(Pbf)-Ala-Gln(Trt)-
Ile-Ala-Ile-Cys(Trt)-Gly-Met-Ser(OtBu)-Thr(OtBu)-
Trp-Ser(OtBu)-OtBu
wherein:
Z is H or N-terminal protecting group Boc or Fmoc.


SEQUENCE LISTING (7 sequences)

SEQ ID NO: 1 - protein, 12 aa, Artificial
  Synthetic fragment for human relaxin analogs
XSWMEEVIKLCG

SEQ ID NO: 2 - protein, 12 aa, artificial
  Synthetic fragment for human relaxin analogs
XELVRAQIAICG

SEQ ID NO: 3 - protein, 4 aa, Artificial Sequence
  Synthetic fragment for human relaxin analogs
MSTW

SEQ ID NO: 4 - protein, 5 aa, Artificial Sequence
  Synthetic fragment for human relaxin analogs
MSTWX

SEQ ID NO: 5 - protein, 17 aa, Artificial Sequence
  Synthetic fragment for human relaxin analogs
RELVRAQIAICGMSTWX

SEQ ID NO: 6 - protein, 29 aa, Artificial Sequence
  synthetic fragment for synthesis of relaxin analogs
XSWMEEVIKLCGRELVRAQIAICGMSTWX

SEQ ID NO: 7 - protein, 29 aa, Homo sapiens
DSWMEEVIKLCGRELVRAQIAICGMSTWS